(19) United States
(12) Patent Application Publication
(10) Pub. No.: US 2008/0058932 A1
(43) Pub. Date: Mar. 6, 2008

(54) Rigidization-on-command orthopedic devices and methods

(75) Inventors: Hai H. Trieu, Cordova, TN (US); Dimitri Protopsaltis, Memphis, TN (US); Jeff R. Justis, Collierville, TN (US); Michael C. Sherman, Memphis, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic Inc.

(21) Appl. No.: 11/493,783

(22) Filed: Jul. 26, 2006

(51) Int. Cl. A61F 2/44

(52) U.S. Cl. 623/17.11

(57) ABSTRACT

Orthopedic devices include a body having an internal chamber housing a curable material. At least a portion of the body provides a barrier member structured to transmit an energy source therethrough to contact the curable material disposed in the internal chamber. The curable material is provided in a first form that provides flexibility to the body and is structured to rigidize in a second form when exposed to the energy source.

### BACKGROUND

The present invention relates to orthopedic devices and manners of using the same, and more particularly, but not exclusively, relates to spinal devices, systems, and implants for treatment of spinal deformities and other conditions.

The use of prosthetic implants to address orthopedic injuries and ailments has become commonplace. In this arena, it is often desired to decrease the invasiveness of the procedures, improve implant integrity, and provide more positive patient outcomes. Particularly, it is often desired to provide an implant with flexible characteristics to facilitate implantation while retaining sufficient rigidity to provide support for corrective treatment. Unfortunately, current devices can be limiting in certain applications. Thus, there is a need for additional contributions in this area of technology.

### SUMMARY

One aspect of the present application is a unique prosthesis having a first state that provides more flexibility than a second state. Other aspects include unique methods, systems, devices, instrumentation, and apparatus involving an orthopedic implantable device.

In one aspect, orthopedic devices include a body having an internal chamber housing a curable material. At least a portion of the body provides a barrier member structured to transmit an energy source therethrough to contact the curable material disposed in the internal chamber. The curable material is provided in a first form that provides flexibility to the body and is structured to rigidize in a second form when exposed to the energy source.

In one aspect there is a spinal device that includes a barrier material structured to transmit an energy source therethrough. The barrier material can comprise at least a portion of a body with an internal chamber. Also included is a curable material disposed within the internal chamber which is structured to rigidize in a second form upon exposure to an energy source. Before the curable material is exposed to the energy source the body has a flexible first form. The curable material cures to provide a rigid body.

In a further aspect, there is provided a spinal device including a photocurable composition. The photocurable composition is housed in liquid form within a body that includes a light transmitting barrier member in communication with the composition. The barrier member is structured to permit light to pass therethrough to expose it to photocurable composition to transform the photocurable composition from a liquid form to a second rigidized configuration.

Still another aspect includes a method for treating a spinal condition. The method includes providing a spinal device comprising a curable material disposed within a body, wherein the curable material is structured to transform from a first flexible configuration to a second rigid configuration when the curable material is exposed to an energy source; flexing the spinal implant for positioning the spinal implant at a desired spinal location with the curable member disposed within the body; and exposing the spinal device to an energy source to transform the spinal implant from the first configuration to the second configuration.

Further embodiments, forms, features, aspects, benefits, objects, and advantages of the present application shall become apparent from the detailed description and figures provided herewith.

### BRIEF DESCRIPTION OF THE DRAWINGS

FIG. 1A is a diagrammatic side plan view of an intradiscal implant device relative to the spinal column of a patient.

FIG. 1B is a sectional view of the implant device of FIG. 1A along view line 1B-1B of FIG. 1A.

FIG. 2 is a perspective view of another embodiment intradiscal implant device with some features shown in phantom.

FIG. 3 is a side plan view of another embodiment intradiscal implant device with some features shown in phantom.

FIG. 4 is a perspective view of another embodiment intradiscal implant device with some features shown in phantom.

FIG. 5 is a perspective view of another embodiment intradiscal implant device with some features shown in phantom.

FIG. 6 is a side plan view of an extradiscal spinal implant system relative to the spinal column of a patient.

FIG. 7A is a side view of a bone anchor device of the spinal implant system of FIG. 6 with some features shown in phantom.

FIG. 7B is a side plan view of an adjustable configuration bone anchor device according to an alternative embodiment of the bone anchor device of FIG. 6, with some features shown in phantom.

FIG. 8A is a perspective view of an elongate spinal fixation element device of the spinal implant system of FIG. 6, with some features being shown in phantom.

FIG. 8B is a cross sectional view of the elongate spinal fixation element device of FIG. 8A taken along view line 8B-8B in FIG. 8A.

FIG. 9 is a side plan view of a crosslink device which may be used with the spinal implant system of FIG. 6 with some features shown in phantom.

FIG. 10 is a plan view of an alternative embodiment of an elongate spinal fixation element device with some features shown in phantom.

FIG. 11 is a cross sectional view of the elongate spinal fixation element device of FIG. 8A further including an energy source.

FIG. 12 is a side plan view of an alternative embodiment of an elongate spinal fixation element device.

FIG. 13 is a cross sectional view of an alternative embodiment of the elongate spinal fixation element device of FIG. 8A.

FIG. 14 is a cross sectional view of an alternative embodiment of the elongate spinal fixation element device of FIG. 8A.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Orthopedic devices are provided that include a body formed at least in part by a barrier material that is in communication with a curable, liquid-like composition housed in the body. The body can be flexible for implantation and then assume a more rigid state when the curable composition is exposed to an energy source that initiates curing of the composition. The curable composition can be self-contained in the body so that injection or handling of the curable material is not necessary during the surgical procedure. It is contemplated that the devices can be intradiscal implant and/or extradiscal implants for spinal stabilization procedures. It is also contemplated that the devices can be employed at other regions of the body.

In one form, the barrier member is energy permeable and structured such that energy from an energy source can pass therethrough to contact the curable material housed in the body of the device and initiate curing thereof. In an exemplary embodiment, when the curable material cures upon exposure to thermal energy, the barrier member may comprise any biocompatible material which conducts heat and is capable of, either alone or in conjunction with the body of the implant, sealingly enclosing the curable material. The curable material in a thermally activated form can be provided commercially, for example, by 3M® corporation as 3M® Scotch-Weld® One Part Epoxy Adhesive.

In another embodiment, the curable material comprises a photocurable material. In an exemplary embodiment when curable material is photocurable, it comprises a pre-activated epoxy adhesive with medium viscosity. One photocurable material of this nature is commercially available by the Henkel Corporation as Loctite® 3355. Among other attributes, this material has single component construction, curability upon exposure to UV light, fast cure time, and low shrinkage and resistance characteristics upon cure. This material also includes low outgasing and will cure evenly across all regions, even those that are shaded. When the curable material takes this form, the barrier member is structured to transmit light therethrough and in one embodiment may be transparent or, in an alternative embodiment, may be translucent. When light is transmitted through the barrier member and contacts the curable material it begins to cure to form a rigid device.

The barrier member material, either alone or in conjunction with other materials comprising the body of the device, prevents contact of the curable material with host tissues at least when the curable material is in a liquid-like form. The barrier material may be biostable or bioresorbable. Examples of suitable barrier material include polyethylene (PE) sheets and tubing, polyethylene terephthalate (PET) sheets and tubing, polyamide balloons, polyurethane pouches, polylactic acid (PLA) or PLDLA sheeting, to name a few. The curable material can cure upon exposure to any one or combinations of energy sources, including thermal energy, mechanical energy, electrical energy, gamma radiation, electron beam radiation, x-ray, ultraviolet light, infrared light, and radio frequency, to name a few. Exposure to initiate curing can be conducted either before or after implantation of the device in the patient.

In one embodiment, a range of time in which the device is exposed to the energy source to initiate and/or complete curing of the curable material is contemplated. In one form, the exposure time ranges from about one second to about 30 minutes. In another form, the exposure time ranges from about 5 seconds to about 5 minutes. After exposure to the energy source, it is further contemplated that the device can be configured to provide a working time in which the device can be worked or manipulated prior to significant curing that increases the modulus to a point where working of the device is not feasible or practicable. The working time can range from about one minute to about 60 minutes in one form. In another form, the working time can range from about 5 minutes to about 30 minutes.

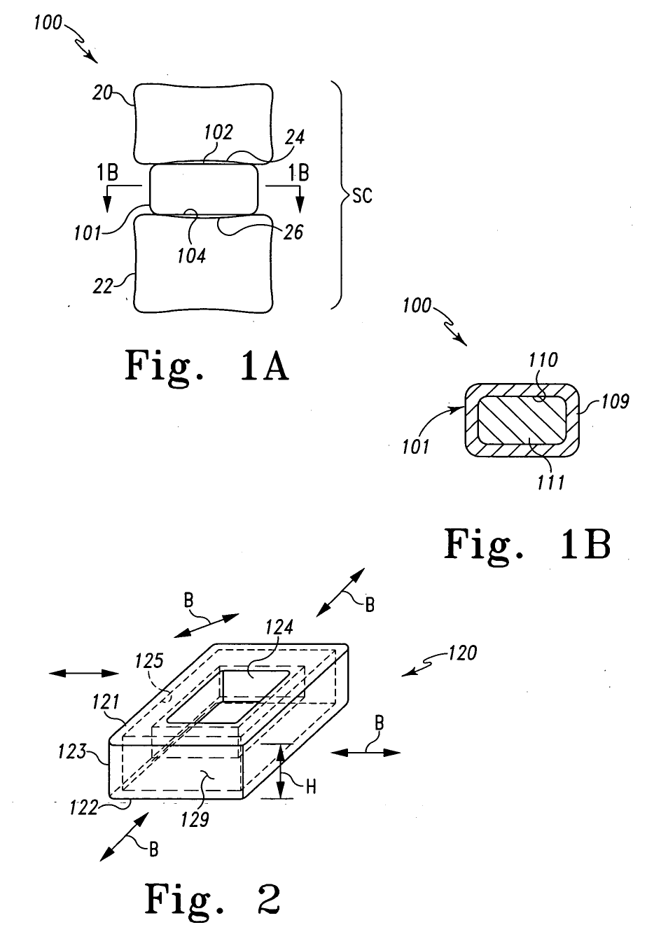

FIG. 1A shows one example of an orthopedic device and is generally directed to an intradiscal spinal implant 100 relative to the spinal column SC of a patient. Implant 100 may be used for treatment of several spinal deformities, including, but not limited to, treatment of degenerative spondylolisthesis, fracture, dislocation, scoliosis, kyphosis, spinal tumor, and/or a failed previous fusion. In the illustrated embodiment, implant 100 is disposed between a first vertebral body 20 and a second vertebral body 22, with each vertebral body including an endplate 24, 26, respectively and wherein endplates 24 and 26 are oriented toward one another. The space between endplates 24, 26 can be formed by removal or all or a portion of a disc space. Additionally, implant 100 can be employed in corpectomy procedures where one or more vertebrae are removed.

Implant 100 includes a first vertebral engaging surface 102 and a second vertebral engaging surface 104 disposed on opposite sides of body 101, wherein each surface 102, 104 is structured to engage an adjacent one of the endplates 24, 26 respectively. Surfaces 102 and 104 are depicted as relatively smooth, but may include alternative surface features in specific embodiments to facilitate engagement with endplates 24 and 26. For example, the structure of surfaces 102 and 104 may be porous and/or include ridges, valleys, spikes, knurling, and/or other securing structures as would be appreciated by one having ordinary skill in the art.

FIG. 1B is a sectional view of implant 100 along view line 1B-1B in FIG. 1A. Spinal implant 100 includes a barrier member 109 about all or a portion of body 101 that forms an internal chamber 110 which houses a curable material 111. Curable material 111 is in communication with barrier member 109 and is sealed in the chamber 110 so that it cannot leak or flow from out of barrier member 109 and/or body 101. However, the provision of one or more ports that are resealable to selectively allow flow of curable material 111 therethrough is not precluded. Each of barrier member 109 and curable material 111 is generally structured such that implant 100 has an initial flexible, bendable or formable configuration. However, when curable material 111 is exposed to an energy source it rigidizes and creates a rigid implant.

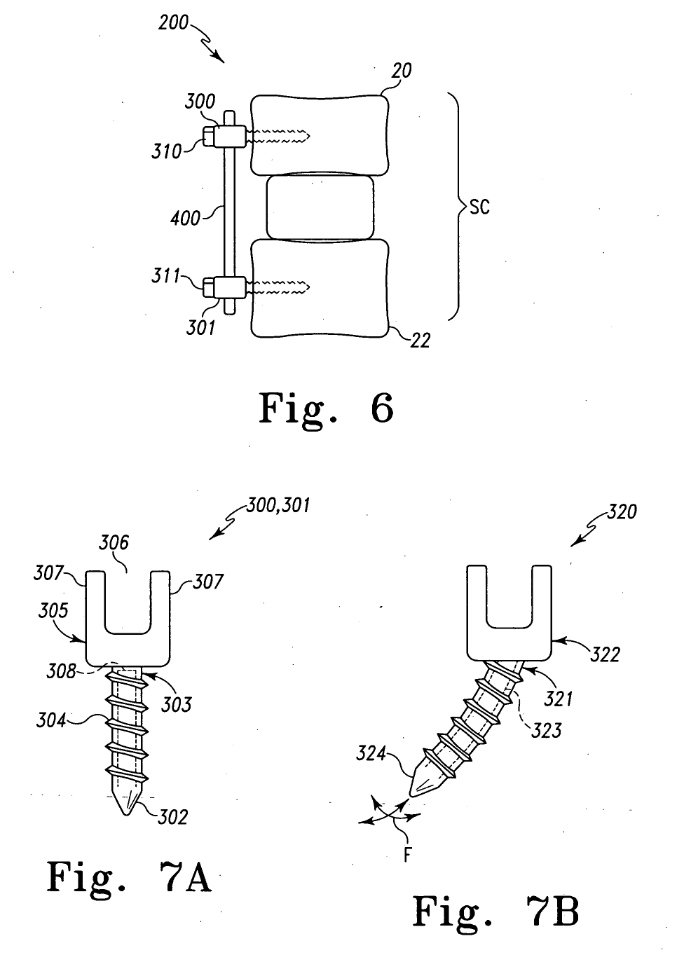

It should be appreciated that each of the implant devices in FIGS. 2-5 can also be intradiscal implant devices and may be used for treatment of the spinal deformities as listed above in regard to spinal implant 100. Additionally, it should be appreciated that each of the devices of FIGS. 1A-S and FIGS. 6-10 include a barrier member surrounding or adjacent to one or more internal chambers, wherein the internal chamber(s) further includes curable material 111 housed therein. Each of the implant devices in FIGS. 1A-5 illustrated are just examples of the many types, shapes, forms and configurations of intradiscal implant devices contemplated by the present invention, while each of the devices in FIGS. 6-10 are just examples of extradiscal implant devices contemplated by the present invention. Furthermore, it should be appreciated that the present invention has applications in other forms, shapes, configurations of other extradiscal implant devices.

Implant 120 is shown is a perspective view in FIG. 2. Implant 20 can be sized and shaped to occupy all or substantially all of a spinal disc space and can be implanted in an anterior, antero-lateral or lateral procedure. Implant 120 includes a first vertebral engaging surface 121 opposite a second vertebral engaging surface 122 and a body 123. Each of surfaces 121 and 122 are structured to engage the endplate of a vertebral body, for example, endplates 24 and 26 in FIG. 1A. As such, each of surfaces 121 and 122 may include securing features such as ridges, valleys, teeth, knurling, and/or other projections or engagement structure. As is known in the art, surfaces 121 and 122 may comprise a porous material to facilitate ingress and egress of spinal tissues to further secure implant 120 at a spinal location and/or to create fusion of adjacent vertebral bodies.

Implant 120 further comprises an opening 124 extending through body 123 from surface 121 to surface 122. In one embodiment, opening 124 may contain one or more biocompatible materials. In another embodiment, opening 124 contains a bioresorbable material such as a bone growth promoting material including, but not limited to, a bone graft material, a bone morphogenic protein (BMP), bone chips, bone marrow, a demineralized bone matrix (DBM), mesenchymal stem cells, and/or a LIM mineralization protein (LMP) or any other suitable bone growth promoting material or substance.

In the illustrated embodiment, the entire body 123 surrounding opening 124 includes internal chamber 125, and all or a portion of body 123 can provide a barrier member 129 in communication with the curable material housed in chamber 125. Internal chamber 125 includes curable material such as curable material 111 discussed above. When the curable material has not been exposed to an energy source, it allows spinal implant 120 to be reconfigured to a multitude of different configurations, as indicated by directional arrows B. For example, among other configurations, all or part of either of surfaces 121 and 122 may be curved to properly seat against a wholly or partially curved spinal endplate, or the height H of the spinal implant 120 may be altered to properly fill a space between intervertebral bodies.

It should be understood that in alternative embodiments not shown only a section or sections of body 123 may contain internal chamber 125 with curable material in order to provide flexibility at certain locations. In these embodiments, the remainder of implant 120 not including internal chamber 125 with curable material may comprise any suitable biocompatible material as would be recognized by one having skill in the art. Additionally, in another embodiment not shown, it is contemplated that only a portion or portions of height H between surface 121 and surface 122 will include internal chamber 125 and curable material such that body 123 takes on a multi-planar configuration, with certain planes comprising curable material 111 while the remaining planes comprise any suitable biocompatible material that is the same as or that is different from the material of barrier 129. In each of the embodiments contemplated, the curable material may be exposed to an energy source to create a rigid spinal implant 120 of a desired configured formation.

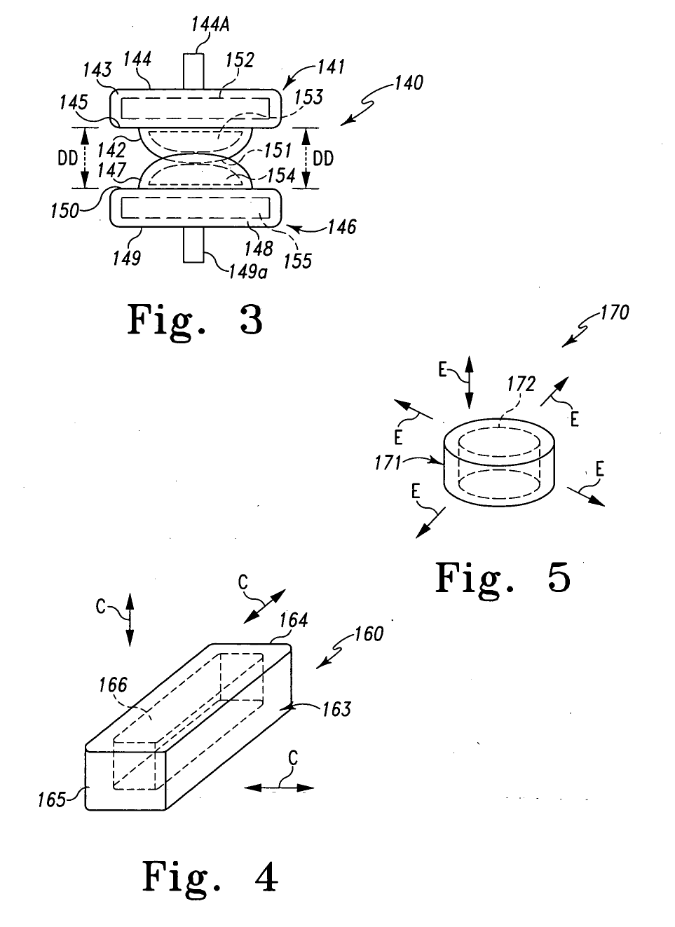

Referring now to FIG. 3, there is illustrated an intradiscal articulating spinal implant 140. Implant 140 includes a first articulating section 141. Section 141 includes an articulating member 142 attached to inner surface 145 of a first mounting plate 143. Implant 140 also includes a second articulating section 146 including an articulating member 147 attached to inner surface 150 of a second mounting plate 148. Each of mounting plates 143 and 148 includes a vertebral engaging surface 144 and 149, respectively. Engaging surfaces 144 and 149 may include one or more bone engagement structures 144a, 149a such as keels as shown. Other bone engagement structures are contemplated including, but not limited to, ridges, valleys, teeth, knurling, and/or other projections or engagement structure(s). It is further contemplated that engaging surfaces 144 and 149 may be porous to promote bone and/or tissue ingrowth into mounting plates 143 and 148 as would be appreciated by one having skill in the art. In another embodiment not shown, mounting plates 143 and 148 may include one or more flanges and/or apertures extending therethrough, wherein the apertures are structured to permit passage of an anchor, including but not limited to, screws, hooks, staples, and/or sutures, to secure implant 140 to each of the respective adjacent vertebral bodies. It should be understood that the addition of apertures and anchor devices may be used alone or in combination with any of the above listed bone engaging structures.

Articulating section 141 and articulating section 146 are structured to engage with one another at interface 151 such that mounting plates 143 and 148 are movable relative to one another. As one having skill in the art would recognize, when implant 140 is implanted into an intervertebral space the articulation between sections 141 and 146 creates a spinal disc-like motion, and as such, implant 140 may be used for disc replacement, among other applications. In the illustrated embodiment, articulating sections 141, 146 are arranged in a ball-and-socket type arrangement. Other embodiments contemplate other arrangements, including resiliently compressible members between mounting plates 143, 148, spring elements between plates 143, 148, or other suitable motion preserving structures.

In the embodiment illustrated, each of first mounting plate 143, articulating member 142, articulating member 147, and second mounting plate 148 is formed of material that houses respective ones of the internal chambers 152, 153, 154, and 155. Each of internal chambers 152-155 further includes a curable material such as curable material 111 such that the implant 140 has an initial flexible configuration provided by the curable material 111 and the structure of the barrier material. In another embodiment, only one of articulating sections 141 or 146 may include one or both of the internal chambers 152 and 153 or 154 and 155 and the associated curable material therein. Still, in another embodiment, one or more of the portions comprising articulating sections 141 and 146 may include its respective internal chamber and curable material 111. For example, one or more of mounting plates 143 and 148 may include internal chamber 152 or 155 and curable material such that one or more of mounting plates 143 and 148 may be configured to matingly engage with the natural or formed surface characteristics of an adjacent vertebral endplate. In another example, one or more of articulating members 142 and 147 includes curable material within the respective internal chamber 153 or 154 such that the one or more of articulating members 142 and 147 is configurable, for example, to change the distance DD between mounting plates 143 and 148 to facilitate insertion into the intradiscal space. It is further contemplated that any of the articulating members 142 and 147 and any of the mounting plates 143 and 148 may include the flexible configuration singly or in combination with any of the other implant components. In each of the embodiments contemplated, the curable material may be exposed to an energy source to create a rigid spinal implant 140 of a desired configured formation.

An implant 160 for a posterior-lateral or posterior interbody fusion procedure is illustrated in a perspective view in FIG. 4. Implant 160 may be used alone or in combination with one or more other implants in a spinal disc space. Implant 160 includes a width to accommodate insertion through a portal created posteriorly or postero-laterally, and can be elongated for orientation in the anterior-posterior directions in the disc space. Other arrangements contemplate implantation in orientations obliquely oriented to the sagittal plane or transversely to the sagittal plane in a transforaminal placement. Alternatively, implant 160 can be implanted anteriorly in side-by-side relation with another implant 160 in an anterior fusion procedure.

Spinal implant 160 includes an elongate body 163 extending between a first end 164 and a second end 165. While body 163 is shown having a substantially elongated rectangular shape and a corresponding rectangular cross section, it is contemplated that other cross section shapes are suitable, for example, including but not limited to, a substantially circular, triangular, hexagonal, or octagonal shape. The upper and lower surfaces can be convexly curved to the endplate anatomy. One or more of the sidewalls can include a concave shape or convex shape. In an embodiment not shown, spinal implant 160 may include external threading extending along all or part of body 163 between ends 164 and 165 to provided threaded engagement between adjacent vertebral bodies. Implant 160 may include other engagement structures along all or a portion of its outer surfaces, including porous structures, ridges, grooves, teeth, and/or other projections, all of which are structured to improve implant holding power and/or fusion at the implant site.

As illustrated, spinal implant 160 includes internal chamber 166 with curable material therein extending along substantially all of body 163. In such an embodiment, body 163 provides a barrier member that encloses internal chamber 166 such that the curable material is contained within body 163. When internal chamber 166 with curable material extends along substantially all of body 163, implant 160 can be configurable in multiple directions as indicated by directional arrows C to change its shape length, width and/or height to accommodate an implantation site or insertion portal. In another embodiment not shown, body 163 includes and internal chamber with curable material that extends along only a section of body 163. The portion of body 163 along the internal chamber is formed at least in part by a barrier member, and the remainder of body 163 can be formed by any suitable biocompatible material. In certain embodiments, body 163 may include at least one cavity structured to contain a bone growth inducing agent such as, but not limited to, a bone graft material, a bone morphogenic protein (BMP), bone chips, bone marrow, a demineralized bone matrix (DBM), mesenchymal stem cells, and/or a LIM mineralization protein (LMP) or any other suitable bone growth promoting material or substance. In each of the embodiments contemplated for implant 160, the curable material in the chamber may be exposed through a barrier member to an energy source to create a rigid spinal implant 160 of a desired configured formation.

FIG. 5 is a perspective view of an intradiscal implant 170 that can be used for either partial or entire replacement of the nucleus pulposus to facilitate augmentation of the annulus fibrosis. Implant 170 includes a body 171 that forms a barrier member which surrounds and creates internal chamber 172 to house curable material therein. In this embodiment, implant 170 is configurable in multiple directions, as indicated by, for example, directional arrows E, which indicate height and radial adjustability of body 171 to better fit an implant site. In another embodiment not shown, implant 170 may include internal chamber 172 in only a selected portion or portions of body 171. Additionally, as illustrated, implant 170 can have a substantially cylindrical shape, but it should be understand that alternative shapes and structures for implant 170 are contemplated. For example, the size, height, and shape of implant 170 may be changed to better conform to the shape of a correspondingly prepared implant site, natural anatomic features, or insertion portal of particular size and shape. In each of the embodiments contemplated, the curable material may be exposed to an energy source to create a rigid spinal implant 170 of a desired configured formation.

Referring now to FIG. 6, there is shown another device that includes an extradiscal spinal implant system 200 in side plan view relative to the spinal column SC of a patient. Spinal implant system 200 includes a pair of anchor devices 300, 301 and an elongate fixation element device in the form of spinal rod 400. Furthermore, as will be appreciated by one having skill in the art, system 200 may include additional components, like for example, a crosslink device 500 as shown in FIG. 9. System 200 may be used for treatment of several spinal deformities, including, but not limited to, treatment of degenerative spondylolisthesis, fracture, dislocation, scoliosis, kyphosis, spinal tumor, and/or a failed previous fusion. As will be described herein, it is contemplated that each of the components of system 200 illustrated in FIGS. 6-10 is just one of the many extradiscal devices to which the present invention may have application.

Referring now to FIG. 7A, wherein each of anchor devices 300 and 301 is shown in side plan view with certain features illustrated in phantom. Anchor devices 300 and 301, respectively, can each have an elongated shaft or stem 303 with bone engaging structures 304. Structures 304 may be in form of threads, spikes, barbs or other structure. A stem without bone engaging structures is also contemplated. Stem 303 is structured to be positioned in and engage a passageway prepared in one or more bones or bony structures in a standard manner, and can be provided with cutting flutes or other structure for self-tapping and/or self-drilling capabilities. Stem 303 can also be cannulated to receive a guidewire to facilitate placement and may further include fenestrations or other openings for placement of bone growth material.

Anchor devices 300, 301 can include a head or a receiver portion 305 defining a receiving channel 306 between upright arms 307. Head or receiver portion 305 can be fixed relative to stem 303 to provide a uni-axial arrangement. Receiving channel 306 is sized and shaped to receive spinal rod 400 and may include structures to engage engaging members 310, 311 for securing spinal rod to head 305, such as internal threading along receiving channel 306 or external threading on head 305, both of which are not shown. In another embodiment, head 305 may include any means for securing spinal rod 400 thereto as would be known to one having skill in the art. As illustrated, receiving channel 306 can be concavely curved and form a passage having a shape of a portion of a circle to receive the rod in form fitting engagement therein. Other embodiments contemplate that the rod is positioned against a proximal head of the stem, or against a cap or crown adjacent a head of the stem, in receiving channel 306. It is further contemplated that receiving channel 306 can be shaped in a variety of configurations to correspond to spinal rod 400 having a non-circular cross section, such as but not limited to, an oval, rectangular, hexagonal, or octagonal cross section.

Referring now to FIG. 7B, another embodiment anchor device 320 is shown. Anchor device 320 can be in form of a stem portion 321 pivotally captured in head portion 322. Pivotal anchor device 320 may be multi-axial, poly-axial, uni-axial, or uni-planar where stem portion 321 and head 322 are movable relative to one another as indicated by directional arrows F. In one movable form, stem portion 321 and head 322 are engaged together with a “ball and joint” or swivel type of coupling that permits relatively universal movement therebetween during at least some stages of assembly.

In yet another form, implant system 200 may include bone anchors in the form of one or more hooks to engage an adjacent bony structure such as a pedicle, lamina, spinous process, transverse process, or other bony structure suitable engaged with a spinal hook. For instance, a multi-axial laminar hook form of a bone anchor can be used in place of one or more of the anchors devices 300, 301. In still other embodiments, the bone anchor can include a bone attachment structure in the form of a staple, bone plate, interbody fusion device, interbody spacer, spinal anchor, intravertebral fusion device, bone clamp, or other anchor.

Anchors devices 300, 301, and 320 each include an internal chamber 308 and 323 respectively. As illustrated in FIG. 7A, internal chamber 308 extends along stem 303 from proximal head 305 to distal tip 302. As illustrated in FIG. 7B, internal chamber 323 extends along stem 321 from proximal head 322 to distal tip 324. Each of internal chambers 308, 323 includes curable material that is enclosed by barrier member formed at least in part by the respective stem 303, 321. In a first configuration, when the curable material has not been exposed to an energy source, stems 303 and 321 remain flexible to facilitate engagement of the stems within a prepared passageway or to allow angular adjustment along the axis of the stems to better facilitate connection with spinal rod 400 or other implant devices.

In one embodiment, the curable material housed in stems 303, 321 is structured such that it may be exposed to an energy source before implantation and remain in the flexible configuration for a period of time post-exposure to facilitate implantation and angular adjustments of stems 303, 321 before becoming rigid. In alternative embodiments not shown, heads 305 or 322 may include an internal chamber with curable material to better facilitate connection of spinal rod 400 or other devices thereto either singly, or in combination with stems 303 and 321 including internal chambers 309 and 323 respectively. For example, arms 307 could be bent around the rod when flexible and cured to rigidly engage the rod in the passage between arms 307. Furthermore, it is contemplated that only a section of stem 303 and/or 321 may include an internal chamber with curable material 111 to control flexibility of the respective anchor device 300, 301, and/or 320.

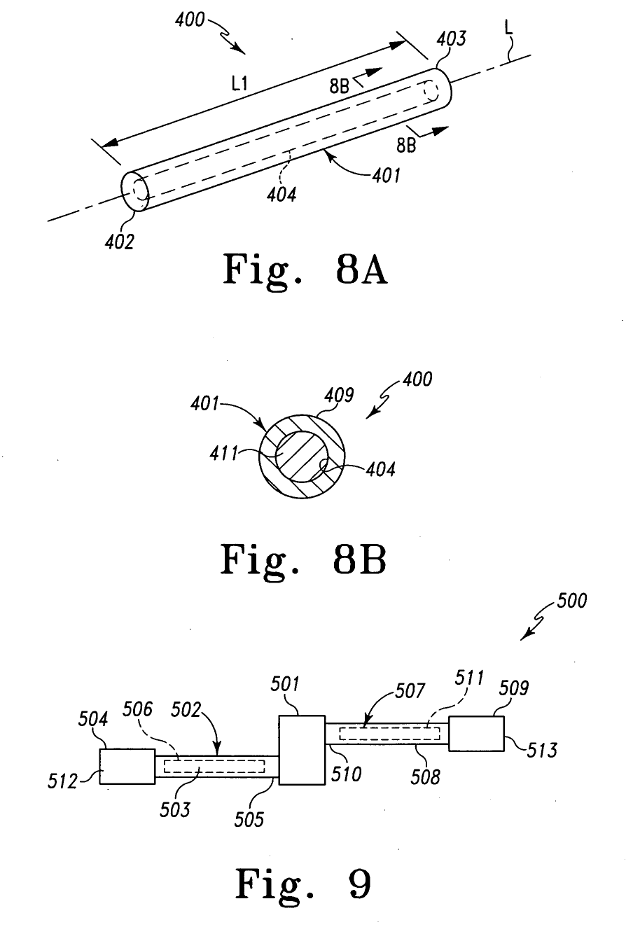

Spinal rod 400 of implant system 200 is illustrated in FIG. 8A. Spinal rod 400 generally includes elongated body 401 extending along longitudinal axis L between first end 402 and second end 403. The length L1 of spinal rod 400 extending between first end 402 and second end 403 is typically great enough to span a distance between at least adjacent vertebral bodies, but in alternative embodiments may have a length L1 sized to span a distance between more or less than two vertebral bodies. As illustrated, spinal rod 400 contains a substantially circular or round sectional profile. It is contemplated however that the sectional profile of spinal rod 400 may vary in alternative embodiments. For example, the sectional profile of spinal rod 400 may include, but is not limited to, triangular, rectangular, hexagonal, octagonal, oval, or star shaped just to name a few possibilities.

Spinal rod 400 is sized and structured to engage with a receiving portion of a bone anchor, for example, receiving channel 306 of bone anchor devices 300, 301, 320 discussed above. When placed in receiving channel 306, spinal rod 400 may be coupled thereto create a rigid construct between two or more bone anchor devices. Spinal rod 400 may also be passively secured to a bone anchor to permit relative motion between the bone anchor and spinal rod 400.

In FIG. 8A spinal rod 400 includes internal chamber 404 extending along a substantial portion of the length L1 of rod 400. Internal chamber 404 is enclosed at least in part by barrier member 409, which may comprise all or a portion of body 401, and houses curable material 411. In the embodiment illustrated, in a first configuration when curable material 411 has not been exposed to an energy source, spinal rod 400 remains flexible along a substantial portion of length L1. In a second configuration, upon exposure to an energy source, curable material 411 cures and spinal rod 400 becomes more rigid or rigid along length L1.

While internal chamber 404 is shown extending along a substantial portion of length L1 of body 401, it should be understood that in alternative embodiments not shown internal chamber 404 may extend along only a portion of length L1. Furthermore, it is contemplated that body 401 may include more than one internal chamber 404 such that spinal rod 400 includes more than one flexible portion while in an initial configuration. In the embodiments where spinal rod 400 includes more than one flexible portion or where internal chamber 404 only extends along a portion of length L1, the remaining section or sections may comprise any suitable biocompatible material including, but not limited to, stainless steel, nitinol, chrome cobalt, titanium and alloys thereof, and polymers. In addition, the structure of the remaining sections of spinal rod 400 may be solid or include cannulations or passages to receive tethers, wires, or cables.

Referring now to FIG. 8B there is shown a cross sectional view of spinal rod 400 viewed along view line 8B-8B of FIG. 8A. As illustrated, the sectional profile of spinal rod 400 is substantially circular and barrier member 409 sealingly encloses curable material 411 to prevent leakage of curable material 411. Curable material 411 is structured to have an initial liquid-like configuration before exposure to an energy source and to transform to a rigid composition subsequent to exposure to an energy source and a cure period. The amount of time required for transition from the flexible configuration to the rigid configuration will be dependent upon the type of material comprising curable material 411. Once in a cured state, the material is structured to provide support for all or part of a respective implant. Furthermore, curable material 411 may be any composition designed to cure upon exposure to various sources of energy as discussed above.

Spinal implant system 200 may further include crosslink device 500, shown in side elevation view with some features in phantom in FIG. 9. Crosslink device 500 includes a first branch member 502 and a second branch member 507 connected at an interconnection device 501. Interconnection device 501 may be structured to facilitate translation and/or rotation of branch members 502 and/or 507 relative to interconnection device 501 and/or each other. Alternatively, branch members 502 and/or 507 can be formed integrally as a single unit with interconnection device 501 or with one another. Branch member 502 includes a body 503 between first end 504 and second end 505. Body 503 includes internal chamber 506 enclosed by body 503 that provides a barrier member, and curable material can be housed within internal chamber 506. Branch member 507 includes first end 509 opposite second end 510 with body 508 extending therebetween. Body 508 includes internal chamber 511 that provides a barrier member and houses curable material.

Each of branch members 502 and 507 includes an engagement portion 512 and 513, respectively, adjacent ends 503, 509. Engagement portions 512 and 513 can be sized and structured to engage with other components of spinal implant system 200. For example, system 200 may include more than one spinal rod 400 connected to an additional set of bone screws 300 and 301, wherein each of spinal rods 400 extend parallel to each other along the spinal column of a patient. In this embodiment, engagement portions 512 and 513 engage with each of the spinal rods 400 such that crosslink device 500 extends transversely therebetween. In alternative embodiments, engagement portions 512 and 513 may be structured to engage with a bone hook, bone screw, or other anchoring device to which the spinal rod is coupled.

As illustrated, internal chambers 506 and 511 extend substantially along all the length of the respective branch members 502 and 507. Crosslink device 500 can remain flexible to facilitate interconnection of crosslink device 500 with various spinal components before the curable material is exposed to an energy source. After exposure to an energy source, the curable material cures and rigidizes branch members 502 and 507 to create a rigid construct between crosslink device 500 and the respective implant components. In alternative embodiments not shown, internal chamber 506 and/or 511 may extend along only a portion of branch members 502 and/or 507 respectively. Additionally, it is contemplated that each of branch members 502 and/or 507 may include more than one internal chamber with curable material, and that the barrier material may form all or a portion of the branch members 502, 507.

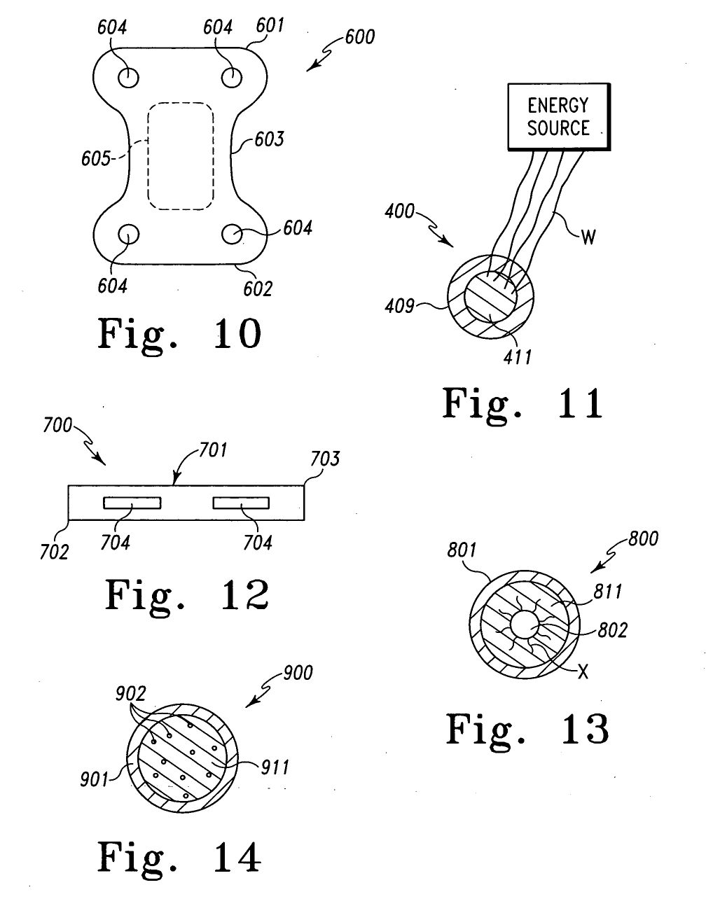

Referring to FIG. 10 there is shown in plan view an alternative elongate spinal fixation element 600 with some features in phantom. Fixation element 600 is in the form of a spinal plate and includes a first end portion 601 opposite a second end portion 602 and includes a body 603 extending therebetween. Fixation element 600 is generally sized and structured to extend between at least one set of adjacent vertebral bodies, but in alternative embodiments may be structured to extend across three or more vertebrae and along one or more regions of the spinal column including the cervical, thoracic, lumbar and sacral regions. Fixation element 600 includes apertures 604 extending through end portions 601 and 602. Apertures 604 are sized and structured to permit passage of an anchoring device, such as a bone screw, to engage with a respective vertebral body so that fixation element 600 may be secured thereto. Furthermore, the exterior of fixation element 600 may include one or more surface features to further promote engagement with a bony structure including intradiscal projections and fusion members, ridges and valleys, and/or a porous material.

Fixation element 600 includes internal chamber 605 within body 603 and between end portions 601 and 602 of device 600. Internal chamber 605 can be sealed by body 603, at least a portion of which provides a barrier member to house curable material and permit passage of an energy source. In a first configuration, body 603 of fixation element 600 remains flexible to facilitate bending and contouring to the spinal anatomy and/or placement through a portal. For example, among other configurations, fixation element 600 may be bent, twisted, flattened, elongated, and/or widened in order to conform to the environmental characteristics of a desired implant location. Once the curable material is exposed to an energy source, fixation element 600 may become rigid in the desired formation.

In alternative embodiments not shown, it is contemplated that one or more of end portions 601 or 602 may comprise an internal chamber with curable material while body 603 extending therebetween comprises a different biocompatible material such that end portion 601 and/or 602 is flexible while in an initial configuration while body 603 is rigid or may be permanently flexible. Additionally, it is contemplated that body 603, either singly or in combination with end portion 601 and/or 602, may include an internal chamber with curable material while the other portion(s) comprise(s) a different material such that body 603 is flexible in an initial configuration while one or both of end portions 601 or 602 is rigid or permanently flexible.

As shown in FIG. 11, spinal rod 400 is viewed in section as shown in FIG. 8B. Barrier member 409 is energy permeable, structured such that energy W from an energy source can pass therethrough to contact curable material 411 and initiate curing thereof. In an exemplary embodiment, when curable material 411 cures upon exposure to thermal energy, barrier member 409 may comprise any biocompatible material which conducts heat and is capable of sealingly enclosing curable material 411. In another embodiment, the curable material comprises a photocurable material that cures when exposed to light through the barrier member. Similar arrangements for can be provided by the other embodiment implants, anchor devices, connection devices and fixation elements discussed herein to cure the curable material housed therein. The barrier member may comprise all or a portion of the respective implant portion surrounding the curable material, so long as the barrier member permits passage of sufficient energy to initiate the curing process.

Referring now to FIG. 12 there is shown a side plan view of an elongate spinal fixation element 700. Fixation element 700 includes a body 701 extending between a first end 702 and a second end 703. Fixation element 700 may comprise a suitable biocompatible material which sealingly encloses curable material. Body 701 further includes one or more portals 704 structured to provide a barrier member and permit communication of energy with the curable material housed in body 701. In one embodiment, portals 704 are comprised of a material different from the rest of body 701 of implant 700 and may comprise an energy permeable material. For example, the material of portals 704 may be in a translucent or transparent form when the curable material is a photocurable composition. In another embodiment not shown, portals 704 may include a cover or a door comprising the same material as the rest of implant 700. The cover or door is moveable to expose curable material housed in body 701 to an energy source. While an elongate spinal fixation element 700 is shown including portals 704, it should be understood that it is contemplated that any of the implants described or contemplated herein may also include one or more portals 704. It should be further understood that portals 704 are structured to transmit energy in any form contemplated herein. Furthermore, the number and/or size of portals 704 may depend on the ability of the curable material to transmit energy through itself to provide uniform curing.

The energy source and energy W will vary in different embodiments dependent upon the composition of the curable material. Furthermore, the curing properties of the curable material might alter with exposure to different energy types. In one embodiment, a spinal implant including curable material may be inserted into an implant site before exposure to energy W. Once implanted and configured to a desired form, the curable material may be exposed to energy W in situ, wherein energy W is transmitted from a hand held or portable energy source. Energy W may be in more than one form when exposure to the curable material occurs. For example, energy W may be UV light and also include thermal energy, which might in some embodiments increase the rate of cure for the curable material. The curable material then rigidizes to provide the implant with a support structure.

In an alternative embodiment a spinal implant including curable material may be exposed to energy W before implantation. In this instance, energy W may come from a portable energy source or the spinal implant may be placed in a chamber transmitting energy W. Once exposed, the spinal implant may be inserted into the implant site and configured as necessary, such that the curable material rigidizes at a desired configuration in situ. When energy W is applied before implantation, it may also contain one or more forms as mentioned above in regard to in situ exposure to energy W. It should be further understood that when desired, a respective implant may be formed and exposed to energy and the curable material allowed to rigidize before implantation of the implant.

It should be further understood that the cure period for the curable material will depend upon the type of material utilized. Furthermore, in certain embodiments, the cure period will be dependent upon the exposure time and intensity of energy W. Additionally, the exposure time and intensity of energy W may depend on the properties of the curable material. For example, in an embodiment where the curable material is a photocurable material, exposure time and intensity may depend on whether the curable material is clear or opaque. It should also be appreciated that the amount of an implant which needs to be exposed, whether in whole or in part, may also depend on one or both of the composition of the curable material and the type of energy W used.

Referring now to FIG. 13 there is shown in cross sectional view an alternative embodiment of a spinal rod 800. It should be appreciated that the external features of spinal rod 800 are similar to that of spinal rod 400 shown in FIG. 8A. Spinal rod 800 includes an exterior shell 801 which may comprise any biocompatible material suitable for use on spinal implants which may also sealingly enclose curable material 811. In one embodiment, exterior shell 801 may be flexible while in other embodiments it is rigid. As illustrated, an energy source 802 is disposed near the center of exterior shell 801 and is surrounded by curable material 811 but may be situated differently in alternative embodiments not shown. Energy source 802 is structured to transmit and expose curable material 811 to energy X upon activation to transform curable material 811 to a rigid form. Activation of energy source 802 may occur before or after implantation of spinal rod 800. It should additionally be understood that energy source 802 may be activated by many ways, including but not limited, electrical activation, remote activation, and/or mechanical activation such as bending or flexing rod 800. Furthermore, it is contemplated that energy source 802 may be capable of transmitting several types of energy, including but not limited to, mechanical energy, electrical energy, light energy, and/or thermal energy. While energy source 802 is shown within spinal rod 800, it should be further appreciated that energy source 802 may be included within the other spinal implants described and contemplated herein.

FIG. 14 illustrates a cross sectional view of a spinal rod 900. It should be appreciated that the external features of spinal rod 900 are similar to that of spinal rod 400 shown in FIG. 8A. Spinal rod 900 includes curable material 911 which is sealingly enclosed by body 901, which provides a barrier member along all or a portion of the length thereof. In this embodiment, spinal rod 900 includes reinforcement members 902. Reinforcement members 902 may comprise, but are not limited to, fused silica, metal or ceramic particles, PET fibers, PET mesh, and/or carbon fibers, just to name a few. Reinforcement members 902 are structured to provide additional implant support to increase implant rigidity and strength when necessary to achieve a desired compression or other force at an implant site. While reinforcement members 902 are shown within spinal rod 900, it should be further appreciated that reinforcement members 902 may be included in the other spinal implants described and contemplated herein.

In another embodiment, spinal rod 900 includes a plurality of energy transmitting members 902. Energy transmitting members 902 may comprise, but are not limited to, embedded fiber optics or other light transmitting material that disperses light or other energy sources throughout the curable material to provide increased uniformity in exposure and curing. In another embodiment, body 901 is formed by light or energy transmitting material so that the energy applied to the device is dispersed throughout the entire or nearly entire surface area of the curable material housed within the barrier member.

The implants provided herein may further include a removable cover or package to protect the implants from exposure to an energy source before an anticipated implant time. The cover may be structured for removal before implantation or may be removed in situ. It is further contemplated that the devices can be provided in a product kit with fully assembled devices that need only to be exposed to an energy source and implanted into the patient either before or after the exposure. In another form, a product kit is provided where the devices are partially assembled or unassembled. In this form, the surgeon can select the device components for assembly during the procedure to provide flexibility in selection in the type of device, the size of the device, and/or the type and amount of curable material with which to fill the device.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A spinal device, comprising:
a body forming an internal chamber and including barrier material structured to transmit an energy source therethrough into said chamber;
a curable material sealed in the internal chamber in a first form to permit manipulation of said body, said curable material is in communication with the barrier material and is structured to rigidize in a second form when exposed to the energy source through the barrier material; and
wherein the body is flexible when the material is in the first form and is rigid when the curable material cures after exposure to the energy source.

2. The spinal device of claim 1, wherein the curable material comprises a photocurable composition.

3. The spinal device of claim 2, wherein the barrier material comprises at least one of a translucent material and a transparent material.

4. The spinal device of claim 2, wherein the barrier material includes at least one light portal.

5. The spinal device claim 1, wherein the curable material comprises a thermally energizeable composition.

6. The device of claim 5, wherein the barrier material comprises a thermal transmitting material.

7. The spinal device of claim 1, wherein the barrier material comprises a polymer material.

8. The spinal device of claim 1, wherein the barrier material comprises a bioresorbable material.

9. The spinal device of claim 1, wherein the body comprises a bone engaging portion opposite a head portion, the head portion being structure to engage an elongated implant element and wherein the bone engaging portion is structured to engage bony tissue.

10. The spinal device of claim 9, wherein the internal chamber extends along the bone engaging portion.

11. The spinal device of claim 9, wherein the bone engaging portion comprises an elongated stem and wherein the head portion is moveable relative to the stem.

12. The spinal device of claim 1, wherein the body is elongated and sized to span a distance between two or more vertebrae.

13. The spinal device of claim 12, wherein the body is in the form of a spinal rod having a generally circular cross-section.

14. The spinal device of claim 13, wherein the internal chamber extends along substantially an entire length of the spinal rod.

15. The spinal device of claim 12, wherein the body includes a plurality of portals therealong formed by the barrier material.

16. The spinal device of claim 1, wherein the body includes a pair of branch members extending oppositely from an interconnection mechanism and an engagement portion at an end of each of the branches engageable with an implant.

17. The spinal device of claim 1, wherein the body comprises a first articulating member including a first endplate and a second articulating member including a second endplate, wherein said first and second articulating members are moveably engaged with one another.

18. The spinal device of claim 17, wherein at least one of the first and second articulating members comprises the internal chamber including the curable material.

19. The spinal device of claim 17, wherein at least one of the first and second endplates comprises the internal chamber including the curable material.

20. The spinal device of claim 1, wherein said body is structured for insertion between adjacent vertebral bodies in contact with endplates of the adjacent vertebral bodies.

21. The spinal device of claim 20, wherein the body includes a cavity between opposite surfaces thereof, the cavity configured to receive bone growth therethrough.

22. The spinal device of claim 1, wherein the body is elongated and structured to extend between at least two or more vertebral bodies, the body further including at least one hole at opposite ends thereof to receive a bone anchor.

23. The spinal device of claim 22, wherein the body is a plate extending between the opposite ends and the internal chamber is located in the body between the at least one holes at the opposite ends.

24. The spinal device of claim 1, wherein the curable material further comprises at least one reinforcement member embedded therein.

25. A spinal device, comprising:
a photocurable composition;
a light transmitting barrier member comprising at least a portion of a body; and
wherein the photocurable composition is disposed within the body in communication with the light transmitting barrier member such that the body houses the photocurable composition in a liquid-like form while allowing light to pass therethrough in order to transform the photocurable composition from the liquid form to a second rigidized configuration.

26. The spinal device of claim 25, wherein the barrier member comprises a transparent material.

27. The spinal device of claim 25, wherein the barrier member comprises a translucent material.

28. The spinal device of claim 25, wherein the barrier member includes at least one light portal.

29. The spinal device of claim 25, wherein the photocurable composition further comprises means for reinforcing the photocurable composition.

30. The spinal device of claim 25, further comprising a plurality of light transmitting elements disposed within the photocurable composition.

31. The spinal device of claim 25, wherein the barrier member forms a sealed enclosure housing the photocurable composition in the liquid form so that the device is bendable before implantation within a patient.

32. A method for treating a spinal condition, comprising:
providing a spinal device comprising a curable material disposed within a chamber of a body with a barrier member in communication with the curable material, wherein the curable material is structured to transform from a first flexible configuration to a second rigid configuration when the curable material is exposed to an energy source;
flexing the spinal device for positioning the spinal device at a desired spinal location with the curable material disposed within the barrier member; and
exposing the spinal device to the energy source to transform the spinal device from the first configuration to the second rigid configuration.

33. The method of claim 32, wherein exposing the spinal device to the energy source occurs prior to flexing the spinal device.

34. The method of claim 32, further comprising positioning the spinal device at the spinal location and wherein exposing the spinal device to the energy source occurs prior to positioning the spinal device.

35. The method of claim 32, further comprising positioning the spinal device at the spinal location and wherein exposing the spinal device to the energy source occurs after positioning the spinal device.

36. The method of claim 32, further comprising positioning the device between vertebrae of a spinal column.

37. The method of claim 36, further comprising movably supporting the vertebrae with the spinal device.

38. The method of claim 36, further comprising fusing the vertebrae.

39. The method of claim 32, further comprising positioning a portion of the spinal device in a passage formed in a vertebra.

40. The method of claim 39, further comprising coupling a spinal rod to the spinal device.

41. The method of claim 32, further comprising engaging the spinal device to anchors engaged to first and second vertebrae.

42. The method of claim 32, further comprising engaging the spinal device to spinal rods extending along the spinal location.

43. The method of claim 32, wherein the curable material comprises a photocurable material and wherein the energy source comprises light.

44. The method of claim 32, wherein the barrier member comprises a light transmitting material.

45. The method of claim 32, further comprising:
a second spinal device comprising a curable material disposed within a chamber of a body with a barrier member in communication with the curable material, wherein the curable material is structured to transform from a first flexible configuration to a second rigid configuration when the curable material is exposed to an energy source;
exposing the second spinal device to the energy source; and
positioning the spinal devices at the desired spinal location in a flexible configuration and wherein the spinal devices transition to a second rigid configuration in situ.

46. The method of claim 32, wherein the energy source is selected from the group consisting of: thermal energy, mechanical energy, electrical energy, gamma radiation, electron beam radiation, x-rays, ultraviolet light, infrared light, and radio frequency.

47. The method of claim 32, wherein the energy source is disposed within the curable material and wherein exposing the spinal device to the energy source further includes activating the energy source in the curable material.

48. The method of claim 32, wherein exposing the spinal device to the energy sources includes exposing the spinal device for a range of time ranging from 1 second to about 30 minutes.

49. The method of claim 32, wherein exposing the spinal device to the energy source includes exposing the spinal device for a range of time ranging from 5 seconds to about 5 minutes.

50. The method of claim 32, wherein after exposing the spinal device to the energy source the spinal device has a working time prior to transforming to the second configuration that ranges from about one minute to about 60 minutes.

51. The method of claim 32, wherein after exposing the spinal device to the energy source the spinal device has a working time prior to transforming to the second configuration that ranges from about 5 minutes to about 30 minutes.
